(19) United States
(12) Patent Application Publication
(10) Pub. No.: US 2025/0359578 A1
(43) Pub. Date: Nov. 27, 2025

(54) WATER-SOLUBLE CREATINE AGGLOMERATE

(71) Applicant: Alzchem Trostberg GmbH, Trostberg (DE)

(72) Inventors: Stephan WINKLER, Altenmarkt (DE); Michael WEGER, Altenmarkt (DE); Barbara NIEß, Trostberg (DE)

(73) Assignee: Alzchem Trostberg GmbH, Trostberg (DE)

(21) Appl. No.: 18/873,906

(22) PCT Filed: Jun. 12, 2023
(86) PCT No.: PCT/EP2023/065664

§ 371 Date: Dec. 11, 2024

(30) Foreign Application Priority Data
Jun. 14, 2022 (DE) 10 2022 114 966.8

(51) Int. Cl. A23L 33/175; A23L 2/39; A23L 2/66; A23L 29/30; A23L 33/125; A23P 10/25; A23P 10/40

(52) U.S. Cl. CPC A23L 33/175; A23L 2/39; A23L 2/66; A23L 29/35; A23L 33/125; A23P 10/25; A23P 10/40

(57) ABSTRACT

The subject matter of the present invention is a creatine agglomerate with improved solubility characteristics in aqueous systems and improved handling, thus simplifying the intake of creatine. The agglomerate is characterized in that it contains 30 to 99.9 wt. % of ground creatine and/or ground creatine derivatives and/or ground creatine salts and 0.1 to 30 wt. % of a binder containing at least one oligosaccharide, in particular maltodextrin, based on the total weight of the agglomerate.

The subject matter of the present invention is a creatine agglomerate with improved solubility characteristics in aqueous systems and improved handling, thus simplifying the intake of creatine.

Creatine is obtained by chemical synthesis. During synthesis, the product is obtained as a crystalline material that can be easily filtered. The creatine obtained has a relatively low solubility in water. For example, the solubility of creatine monohydrate in water at 20° C. is 13 g/L. In addition, complete dissolution of creatine is often only achieved with a delay or not at all in practical applications due to its crystalline condition. This proves to be particularly disadvantageous if the crystalline creatine is taken after being dissolved in a liquid. A residue often remains, which makes the intake unpleasant for the consumer.

A number of “micronized” creatine products are available on the market, the average grain size (x50) of which usually varies between 2 μm and 70 μm. Application WO 2007/095734 A1, for example, discloses a method for improving the bioavailability of dietary supplements, including creatine. For this purpose, the use of micronized dietary supplements with reduced particle size is recommended, which, among other things, have improved dissolution properties in water.

Although the solubility characteristics of micronized creatine products are improved compared to coarsely crystalline products, it is still not ideal. In addition, the handling properties of micronized products typically correspond to those of finely ground products. These products generally have a low bulk density and poor flow and pouring properties, which is disadvantageous in practical use. If the powders are too fine, wettability of the creatine with water suffers, so that the creatine powder is difficult to stir in and therefore difficult to dissolve in water.

Various suggestions have already been made to further improve the solubility properties of creatine and thus the oral intake of creatine using aqueous liquids.

In patent application CN 104 432 095 A, spray-dried creatine particles are described, whereby a creatine suspension is used for spray drying. Large quantities of spray-drying and instantizing additives are required to produce the suspension, and the amount of creatine in the produced granules is correspondingly low.

Patent specification U.S. Pat. No. 9,445,622 B2 describes a method for improving the solubility of nitrogenous organic acids, such as creatine. For this purpose, the addition of certain proteins to ground creatine is suggested.

In US 2002-0151593 A1 creatine monohydrate formulations with improved water solubility are described. For this purpose, creatine monohydrate with an average particle diameter of at most 40 μm and an anti-agglomeration agent, such as dextrose, are mixed and ground.

WO 2017/106687 A1 discloses a method for increasing muscle protein build-up in mammals by administering essential amino acids, amino acid derivatives and nitrogenous organic acids. The administered compositions may contain polysaccharides as binders, among others.

WO 94/17794 A1 describes pharmaceutical blends of glycine derivatives and sugars, including maltodextrin, which can be administered in aqueous solution, among other things.

Despite the improvements achieved to date in the solubility of creatine products, there is still a great demand for products that can be easily incorporated into aqueous liquids, i.e. that are easy to handle and dissolve quickly and reliably. A further aim of the invention is to ensure an as high as possible creatine content in the product without affecting the solubility or handling of the product.

Furthermore, the present invention is based on the problem of finding a dosage form for creatine that requires as few additives as possible.

The problem is solved with finely ground creatine, which is agglomerated after the addition of a binder containing at least one oligosaccharide.

Thus, the subject matter of the present invention is an agglomerate containing
a) 30 to 99.9 wt. % of ground creatine and/or ground creatine derivatives and/or ground creatine salts, based on the total weight of the agglomerate, and
b) 0.1 to 30 wt. % of a binder containing at least one oligosaccharide, based on the total weight of the agglomerate.

Binders are additives whose addition can be helpful for agglomeration in order to increase the stability of the agglomerated particles. For the agglomerates described herein, binders based on oligosaccharides have proven to be suitable for increasing the stability of the creatine particles and at the same time their solubility characteristics in aqueous liquids. On the other hand, such binders also significantly improve the handling of the creatine agglomerates, which are to be dissolved as bulk material in aqueous media.

In addition to the oligosaccharides, binder b) may also contain monosaccharides (simple sugars) and polysaccharides, in particular if it is produced by partial hydrolysis from naturally occurring polysaccharides, as is the case, for example, in the production of maltodextrin from starch.

Preferably, the claimed agglomerate comprises 1 to 20 wt. % of binder b), particularly preferably 3 to 18 wt. %, in particular 5 to 15 wt. %.

Binders b) consist essentially of a carbohydrate selected from the group of oligosaccharides or a mixture of carbohydrates from monosaccharides, oligosaccharides and/or polysaccharides, wherein the carbohydrate or carbohydrates preferably constitute at least 90 wt. %, more preferably at least 95 wt. % and in particular at least 99 wt. % based on the total weight of binder b).

The average molecular weight M_{w} (weight average) of preferred carbohydrate mixtures which can be used as binder b) is in the range from 5,000 to 250,000 g/mol, in particular between 9,000 and 150,000 g/mol and particularly preferably between 12,000 and 100,000 g/mol, even better between 15,000 and 75,000 g/mol. The average molecular weight Mₙ (number average) of preferred carbohydrate mixtures is in the range from 500 to 10,000 g/mol, in particular between 1,250 and 7,500 g/mol and particularly preferably between 1,500 and 6,000 g/mol, even better between 1,500 and 5,000 g/mol. The weight average or number average of the molecular weight can be determined by size-exclusion chromatography as described in Avaltroni F. et al, Carbohydrate Polymers 58 (2004), 323-334 under item 2.4.

Oligosaccharides in the sense of the present invention are preferably polysaccharides with 2 to 15 sugar units, particularly preferably with 3 to 10 sugar units, in particular 3 to 6 sugar units, which are linked to one another via glycosidic bonds. The respective longer-chain polysaccharides are considered to be polysaccharides in the sense of the present invention. Typically, polysaccharides, such as starch, can contain molecules with up to 20,000 sugar units or more. As far as the disaccharides are no longer to be classified as oligosaccharides according to the preferred oligosaccharide definition, they are referred to as disaccharides.

The proportion of simple sugars, such as glucose, should be below 25 mol %, particularly preferably between 0.1 and 15 mol %, in particular between 1 and 10 mol % based on the total weight of the carbohydrate mixture of binder b). The molar proportion of disaccharides, such as maltose, in the carbohydrate mixture should preferably be below 30 mol %, in particular between 1 mol % and 25 mol %, particularly preferably between 5 mol % and 20 mol %. The molar proportion of oligosaccharides with 3 to 6 sugar units in the carbohydrate mixture is preferably above 20 mol %, in particular in the range from 25 mol % to 80 mol %, particularly preferably in the range from 30 mol % to 70 mol %. The proportion of higher oligosaccharides and polysaccharides is preferably below 60 mol-%, in particular between 10 mol-% and 55 mol-%, particularly preferably between 20 mol-% and 50 mol-%.

The sugars of which the oligosaccharides or polysaccharides are composed are preferably hexoses, such as aldohexoses, in particular glucose, mannose and galactose, or ketohexoses, such as fructose, or pentoses, such as ribose or arabinose. The sugars can be present in their D or L configuration or as a mixture of both configurations. Oligo- and polysaccharides which are composed of more than 90 wt. % of hexoses or consist of hexoses are particularly preferred. The sugar units of the oligo- and polysaccharides are preferably linked to one another via glycosidic bonds. Oligo- and polysaccharides containing 50% or more or even better at least 80% glucose units as components are particularly preferred. Gluco-oligosaccharides which are composed exclusively of glucose units, such as maltodextrin, are particularly suitable.

The use of maltodextrin as a binder for creatine agglomerates has proven to be particularly preferred. Maltodextrin is a water-soluble mixture of carbohydrates that is usually produced by partial hydrolysis of starch (poly-α-glucose). The starch for this can originate from cereals or vegetables, e.g. corn, potatoes or tapioca. Hydrolysis can be carried out for example with acid or by enzymatic means or by a combination of both processes.

Maltodextrin is a mixture of monomers, oligomers and polymers of glucose. The composition of the mixture differs depending on the degree of hydrolysis. The mixture is usually described by the dextrose equivalent. According to the invention described herein, products whose dextrose equivalent is between 3 and 20 are referred to as maltodextrin. The creatine agglomerates disclosed herein preferably comprise maltodextrins with a dextrose equivalent of from 3 to 15 preferably, particularly preferably the dextrose equivalent is in the range from 4 to 12, in particular from 4 to 10.

The dextrose equivalent of a polysaccharide mixture is the percentage by mass of reducing sugars (calculated as glucose) in the dry substance. It therefore corresponds to the mass of glucose (=dextrose) that would have the same reducing capacity per 100 g of dry substance. The DE value is a measure of the extent to which starch degradation has taken place, so products with a low DE value have a high proportion of polysaccharides and a low content of low-molecular sugars, while products with a high DE value consist mainly of low-molecular sugars.

The dextrose equivalent (DE) is usually specified by the manufacturers of maltodextrin. However, the DE indication can also be determined by Lane-Eynon titration (Lane, J. H. and Eynon, L., J. Soc. Chem. Ind. Trans. 42 (1923), 32-36) according to DIN EN ISO 5377-1994.

In a particularly preferred embodiment of the present invention, 0.5 to 20 wt. %, preferably 0.5 to 18 wt. %, in particular 1 to 15 wt. % or 5 to 12 wt. % of maltodextrin, based on the total weight of the agglomerate, is used as binder b).

Particularly preferred maltodextrins contain less than 5 wt. %, in particular 0.05 to 3 wt. % of glucose as a simple sugar and less than a maximum of 20 wt. %, preferably between 0.1 and 15 wt. %, in particular between 0.5 and 10 wt. % of maltose. The percentages by weight refer to the total weight of the carbohydrate mixture of binder b).

The agglomerate according to the invention contains 0.1 to 30 wt. % of a binder containing at least one oligosaccharide, based on the total weight of the agglomerate. According to the invention, all carbohydrates contained in the agglomerate, in particular all monosaccharides, oligosaccharides as well as polysaccharides, are preferably assigned to binder b). Particularly preferably, the binder comprises at least 95 wt. %, more preferably at least 99 wt. % and even more preferably at least 99.9 wt. % and in particular exclusively (i.e. 100 wt. %)

carbohydrates, i.e. monosaccharides, oligosaccharides and polysaccharides. In a particularly preferred embodiment, further additives, if present, are not assigned to binder b), but to the further additives c).

Binder b) can consist entirely of oligosaccharides. In this case, the proportion of oligosaccharides in binder b) is 100 wt. %. However, it is also possible that binder b) contains other carbohydrates, in particular monosaccharides or polysaccharides, in addition to the oligosaccharides.

In a preferred embodiment, the proportion of oligosaccharide in binder b) is preferably at least 0.1 wt. %, more preferably at least 0.5 wt. %, and even more preferably at least 1 wt. %, based on the total weight of binder b). More preferably, the proportion of oligosaccharide in binder b) is at least 10 wt. %, even more preferably at least 20 wt. % and most preferably at least 30 wt. %, based on the total weight of binder b). The proportion of oligosaccharide in binder b) can be up to 100 wt. %, preferably up to 99 wt. %, more preferably up to 90 wt. %, even more preferably up to 80 wt. % and most preferably up to 70 wt. % and in particular up to 50 wt. %

Particularly preferably, binder b) consists of ≥ 90 wt. % of carbohydrates, of which in turn 10 to 90 wt. %, preferably 20 to 80 wt. % and even more preferably 30 to 70 wt. % are oligosaccharides.

In a further preferred embodiment, the proportion of oligosaccharide in binder b) is preferably at least 0.1 mol %, more preferably at least 0.5 mol %, and even more preferably at least 1 mol %, based on the total binder b). More preferably, the proportion of oligosaccharide in binder b) is at least 10 mol %, even more preferably at least 20 mol % and most preferably at least 30 mol %, based on the total binder b). The proportion of oligosaccharide in binder b) can be up to 100 mol %, preferably up to 99 mol %, more preferably up to 90 mol %, even more preferably up to 80 mol % and most preferably up to 70 mol %, based on the total binder b).

Particularly preferably, binder b) consists of ≥ 90 wt. %, in particular at least 99 wt. %, of carbohydrates, of which in turn 10 to 90 mol %, preferably 20 to 80 mol % and even more preferably 30 to 70 mol % are oligosaccharides. Creatine is an endogenous substance that plays a central role in the energy metabolism of cells. Creatine can be produced in the body through biosynthesis or supplied through food. The common form in which creatine is supplied as a dietary supplement includes, besides pure creatine, also creatine derivatives, such as creatine hydrates, in particular creatine monohydrate. However, also creatine salts, such as creatine citrate, -pyruvate, -hydrochloride, -hydrobromide, -hydrogen citrate, -maleate, -malate, -nitrate, -mesylate, -dihyrogen phosphate, -hydrogen oxalate, -fumarate, -tartrate, -lipoate, -bicarbonate and -ascorbate are used in dietary supplements.

According to the present description, the term creatine should be understood to include derivatives and salts of creatine in addition to pure creatine, unless explicitly stated otherwise. Thus, the term creatine agglomerate also includes agglomerates of creatine derivatives and creatine salts. As pure creatine is hygroscopic, creatine is preferably used as a hydrate, wherein creatine monohydrate is usually present at equilibrium with humidity.

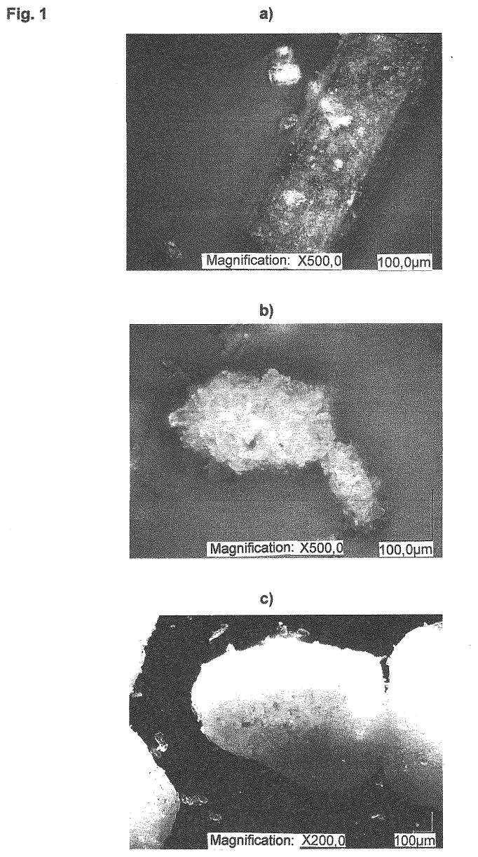

Creatine monohydrate is usually produced by chemical synthesis and is produced as a colorless, crystalline solid (FIG. 1a). The solubility of creatine monohydrate in water is 13 g/L at 20° C. As a dietary supplement, creatine monohydrate is often offered as a powder that is taken in by dissolving it in an aqueous liquid, e.g. mineral water or juice. A disadvantage of conventional creatine monohydrate powders is that they dissolve very slowly due to the low solubility of crystalline creatine monohydrate and often leave a residue. Although creatine monohydrate dissolves relatively well when ground, the ground powders have poor wetting properties and handling needs to be improved. The poor wetting can be recognized by the fact that the creatine powder sinks into the liquid only slowly and/or with aggregate formation on contact with the liquid surface. It is often observed that the aggregates formed in this way dissolve extremely slowly. In addition, the handling of fine powders presents further difficulties, as they often do not flow freely and are difficult to fill or decant. Due to the high dustiness of the powder, it is also difficult to pour the entire powder into a glass, for example, without the suspended particles drifting away. The bulk properties of creatine powder are also not ideal, parts of the powder easily clump together and stick to the packaging material, the angle of repose is high and the flowability and pourability is low.

Other creatine salts, creatine derivatives and pure creatine show a similar behavior.

The disadvantages described can be overcome by agglomeration of ground creatine in the presence of an oligosaccharide-containing binder, such as maltodextrin.

Thereby, agglomerates are formed from the ground creatine particles, the structure of which differs significantly from the crystalline particles of the unground creatine (FIGS. 1b) and c)).

With the described binders, a high content of ground creatine in the agglomerates can be ensured without a significant impairment of the solubility characteristics compared to the direct use of ground creatine powder. This was not to be expected, as the binders have a very good adhesive effect during agglomeration, which should counteract dissolution in aqueous liquids. The handling parameters described are also significantly improved compared to simply ground creatine.

When using starch as a binder, very stable creatine agglomerates are obtained, but the solubility characteristics deteriorate significantly. When dextrose is used as a binder, a moist agglomerated creatine can be produced, but this disintegrates again during drying. The binding properties of dextrose are not sufficient for the production of creatine agglomerates. According to the invention, these disadvantages were eliminated by using a binder comprising at least one oligosaccharide.

Ground creatine is used to produce the creatine agglomerates according to the invention. Ground creatine is preferably characterized by a grain size distribution with an x50 value in the range from 2 μm to 150 μm, an x10 value in the range from 0.01 μm to 20 μm and an x90 value in the range from 15 μm to 250 μm. Preferably, the x50 value of the ground creatine is in the range from 3 μm to 80 μm, particularly preferably between 5 um and 50 um and even more preferably between 5 μm and 30 μm. Preferred x10 values are in the range from 0.1 μm to 10 μm, in particular between 0.5 μm and 5 μm. Preferably, the x90 value is in the range from 20 μm to 100 μm, particularly preferably from 30 μm to 70 μm. The values x10, x50 and x90 are each based on the mass fraction of the respective particle group in the ground creatine. This means that the particles with a particle size above the x50 value constitute 50 wt. % of the agglomerate, the remaining 50 wt. % of the agglomerate comprises particles with a particle size with a value smaller than x50. Accordingly, the agglomerate contains 10 wt. % of particles with a particle size below the x10 value and 10 wt. % above the x90 value.

Particularly preferred ground creatine powders have an x98 value in the range from 50 μm to 300 μm, in particular between 60 and 120 μm. The x98 value is also based on the mass fraction in the ground creatine powder.

The amount of ground creatine in the agglomerate is preferably more than 45 wt. %, particularly preferably at least 60 wt. %, more preferably more than 60 wt. %, even more preferably at least 75 wt. %, in particular more than 80 wt. % based on the total weight of the agglomerates. The upper limit is 99.9 wt. %, preferably 99 wt. %, in particular 95 wt. %.

In a preferred embodiment, the invention relates to an agglomerate comprising
a) 30 to 99.9 wt. %, preferably at least 45 wt. %, more preferably at least 60 wt. % and even more preferably at least 75 wt. % of ground creatine and/or ground creatine derivatives and/or ground creatine salts, in particular creatine monohydrate, based on the total weight of the agglomerate; and
b) 0.1 to 30 wt. %, preferably 1 to 18 wt. % and more preferably 5 to 15 wt. % of a binder containing at least one oligosaccharide, in particular maltodextrin, based on the total weight of the agglomerate; and
c) 0 to 20 wt. %, preferably at most 10 wt. %, more preferably at most 5 wt. % of other additives; and
d) 0 to less than 5 wt. %, preferably up to less than 2 wt. % of free water.

Components a), b), c) and d) preferably constitute 100% of the agglomerate, i.e. the agglomerate does not contain any other substances.

In a preferred embodiment, the invention relates to an agglomerate comprising
a) 30 to 99.9 wt. %, preferably at least 45 wt. %, more preferably at least 60 wt. % and even more preferably at least 75 wt. % of ground creatine and/or ground creatine derivatives and/or ground creatine salts, in particular creatine monohydrate, based on the total weight of the agglomerate; and
b) 0.1 to 30 wt. %, preferably 1 to 18 wt. % and more preferably at most 5 to 15wt. % of a binder containing at least one oligosaccharide, in particular maltodextrin, based on the total weight of the agglomerate; and
d) 0 to less than 5 wt. %, preferably up to less than 2 wt. % of free water.

Components a), b) and d) preferably constitute 100% of the agglomerate, i.e. the agglomerate does not contain any other substances. In a particularly preferred embodiment, the agglomerate according to the invention comprises
a) 30 to 99.9 wt. %, in particular 90 to 99.9 wt. % and particularly preferably 95 to 99.9 wt. % of ground creatine and/or ground creatine derivatives and/or ground creatine salts, in particular creatine monohydrate, based on the total weight of the agglomerate, and
b) 0.1 to 30 wt. %, preferably 1 to 18 wt. % and particularly preferably 5 to 15 wt. % of a binder containing at least one oligosaccharide, based on the total weight of the agglomerate, the binder being particularly preferably maltodextrin; and
d) up to a maximum of 1 wt. % free water.

The particularly preferred agglomerate according to the invention does not contain any other substances or additives.

The agglomerates according to the invention preferably have the following composition:
a) 60 wt. % to 99.9 wt. %, preferably 75% to 99 wt. %, based on the total weight of the agglomerate, of ground creatine, in particular creatine monohydrate;
b) 0.1 wt. % to 30 wt. %, preferably 1 wt. % to 25 wt. %, even more preferably 5 to 15 wt. %, based on the total weight of the agglomerate, of binder containing at least one oligosaccharide; and
c) 0 wt. % to 10 wt. %, in particular up to 5 wt. % of further additives.

Apart from the water bound to creatine as hydrate, as little free water as possible should be present in the agglomerate, as the agglomerate tends to clump together if the moisture content is too high. Preferably, the proportion of free water in the agglomerate should be less than 5 wt. %, particularly preferably between 0.01 and 2 wt. %, in particular between 0.05 and 1 wt. %.

Generally, agglomeration also allows the addition of further additives. Further additives c) include, for example, other binders that are not included in binders b); additives to improve solubility; antioxidants, buffers, sweeteners, colorants and flavorings, proteins, amino acids, vitamins, minerals, trace elements, etc.

Other binders and solubility enhancers to be included under additives c) are, in particular, water-soluble substances that are approved as food additives or pharmaceutical additives, such as polyvinylpyrrolidone (PVP); propylvinyl alcohol; sugar alcohols, including sorbitol, xylitol; amino acids and glycerol; or mixtures of these substances. The minerals include in particular inorganic salts, such as sulphates, chlorides, carbonates, hydrogen carbonates, acetates, citrates, gluconates, ascorbates, pantothenates, lactates, in particular their sodium, potassium, calcium and magnesium salts, as well as mixtures thereof. Particularly preferred are trimagnesium dicitrate, magnesium hydrogen citrate, sodium chloride (common salt), sodium sulphate, sodium acetate, sodium citrate, sodium gluconate, sodium ascorbate, sodium pantothenate and sodium lactate or mixtures of these salts.

If required, the agglomerates can contain a buffer, whereby buffer systems are preferably selected which, when dissolved in an aqueous liquid, adjust the pH value of the liquid to 7 to 12, preferably to 9 to 11. Buffer systems consisting of a combination of a weak acid and a corresponding base, for example, are suitable for this purpose.

Suitable buffer systems are, for example, mixtures of sodium hydrogen phosphate and sodium phosphate or L-lysine and L-lysine sodium salt or L-arginine and L-arginine sodium salt. A mixture of sodium carbonate and sodium hydrogen carbonate is particularly preferred as a buffer system.

The use of buffer systems that create an alkaline environment is preferred, as this increases the stability of creatine against acids and thus prevents the breakdown of creatine in the stomach. Furthermore, sodium ions in particular improve the uptake of creatine into the cells, whereby this effect can also be enhanced by adding further sodium salts.

Sweeteners that are included under additives c) are sweeteners that do not contain carbohydrates. Conventional carbohydrate-based sweeteners, in particular natural sweeteners such as glucose but also fructose, can be contained in binder b), so that the agglomerates described can already have sufficient sweetness. If required, however, further, preferably water-soluble sweeteners such as cyclamate, aspartame, acesulfame, sucralose, stevia or sugar alcohols such as sorbitol, xylitol, mannitol, erythritol or lactitol can be added to the agglomerates. This can be particularly advantageous if the content of simple sugars in the binder is low.

However, simple sugars, such as glucose and fructose, can be mixed with the finished agglomerate in order to achieve sufficient sweetness in the mixture. The latter can be advantageous, since the content of simple sugars in binder b) cannot be increased arbitrarily without impairing handling of the agglomerate.

In particular, compounds that are sufficiently soluble in water are used as colorants and flavorings. Both natural, nature-identical and artificial flavorings can be used, preferably those that are approved for food or pharmaceuticals. Lemon or orange flavoring, for example, but also citric acid or tartaric acid or their salts can be used as flavoring agents. Bitter substances such as quinine or ginger extracts are also considered flavorings.

Vitamins and trace elements can also be added to the agglomerates, in particular those with good water solubility. Preferably, vitamins from the group of vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid), vitamin B12 (cobalamin), vitamin C and vitamin D3 (cholecalciferol) or a mixture thereof can be added to the agglomerates. Trace elements are preferably used in the form of their water-soluble salts and are preferably selected from the group zinc, selenium, molybdenum, manganese, copper, iodine, fluorine, iron and chromium or mixtures thereof.

Additives c) are preferably added during the agglomeration process so that the additives are part of the agglomerates.

In a particular embodiment, the creatine agglomerates described contain no further additives c), in particular no suspension stabilizers from the group of celluloses, gelatins and gum resins, such as microcrystalline celluloses, carboxymethyl celluloses and xathan gum, gellan gum and gum arabic.

The total amount of additives c) in the agglomerate is preferably less than 20 wt. %, in particular less than 10 wt. %, based on the total weight of the agglomerate.

The compounds described as additives c) can also simply be added to the creatine agglomerate if required, provided they do not impair the handling and water solubility of the products too much. In the case of admixture, higher proportions of these substances can also be taken in together with the creatine agglomerate. In addition to the additives c), non-agglomeratable compounds can also be easily added. As already mentioned, mono-and disaccharides can also be mixed with the agglomerate to adjust the sweetness of the mixture. Another important group of additives that can be easily added are flow additives, such as SiO₂.

The proportion of added substances should preferably be less than 80 wt. %. The mixtures usually contain between 10 and 70 wt. %, particularly preferably between 20 and 60 wt. % of such additives, based on the total weight of the mixture, in particular if they are used as beverage powders. Accordingly, such mixtures comprise at least 20 wt. % or more of the creatine agglomerate, in particular between 30 and 90 wt. % and most preferably between 40 and 80 wt. %, based on the total weight of the mixture.

Agglomeration is understood herein as the process of grain enlargement of particulate creatine, wherein small creatine particles in the presence of the binder form larger, permanently agglomerated creatine-containing agglomerates in which the original particles can usually still be identified.

Various processes can be used to agglomerate the ground creatine powder. Agglomeration in the fluidized bed, agglomeration by granulation in a mixer, e.g. an intensive mixer, and agglomeration by extrusion, in particular moist extrusion, have proven to be advantageous.

Consequently, for the purposes of the present invention, the term “agglomeration” also includes the production of agglomerated particles by granulation and extrusion. In addition, other common agglomeration processes are also included, such as agglomeration in the fluidized bed. Accordingly, the term “agglomerate” also includes granulates, fluidized bed agglomerates and extrudates.

Spray agglomeration of creatine in the presence of a binder based on oligosaccharides in the fluidized bed produces irregularly shaped, porous agglomerates. They have good flowability and pourability, are sufficiently abrasion-resistant and have a medium bulk density. In water, they exhibit very uniform wetting behavior, which causes the agglomerates to disintegrate immediately and the primary particles to dissolve in a very short time.

Stable agglomerated extrudates can also be produced by extrusion with the addition of the oligosaccharide-containing binder. If a suitable amount of binder is used, compact, very abrasion-resistant and uniform extrudates are obtained.

It has been shown that the granules and extrudates also dissolve very quickly in liquids, e.g. water, despite their high stability and partly compact condition.

The amount of binder required in the production of the agglomerates is relatively small. For example, between 0.5 wt. % and 15 wt. % of maltodextrin in an agglomerate consisting essentially, i.e. 90 wt. % or more, of binder b) and creatine, e.g. in the form of creatine monohydrate, is often sufficient to agglomerate the creatine.

Suitable methods for producing the agglomerates described are, for example, dry and wet agglomeration. In dry agglomeration, the particles are condensed by pressure in the presence of the binder to form compressed shapes without the addition of liquid and then often crushed to a defined grain size. In wet agglomeration, liquid, usually water or a binder dissolved in water, is added during the agglomeration process, which increases the interfacial forces between the particles in the dried agglomerate and thus stabilizes the structure.

In all of these processes, it has proven to be particularly advantageous to use a process that is as gentle as possible so that the chemical purity of the creatine is maintained, and no undesirable foreign substances are introduced. It has also been shown that the agglomerates are already sufficiently stable if only a binder, which is essentially composed of carbohydrates, is added and no or only a small amount of water of less than 5 wt. %, based on the total weight of the agglomerate, is contained in the product.

The grain size distribution, also known as particle size distribution, indicates the frequency distribution of particle diameters. Various parameters can be read from the distribution function. For example, the x10 value indicates the particle diameter below which 10% of the particles fall. The x50 value indicates the average particle size or the particle diameter below which 50% of the particles fall. Similarly, 90% of the particles are smaller than the x90 value. A creatine agglomerate according to the invention with good properties preferably has a low proportion of fines, represented by the x10 value, which should be at least 1 um or more, in particular in the range between 5 μm and 800 μm, preferably between 10 μm and 600 μm. Furthermore, the average grain size, represented by the x50 value, should be at least 30 μm and the upper grain size, represented by the x90 value, should not be more than 1800 μm. Average grain sizes x50 of 50 μm to 1200 μm or 70 μm to 1000 μm are preferred. The x90 value is preferably between 100 μm and 1500 μm, in particular over 200 μm.

Typically, the difference between the values x90 and x10 is between 100 μm and 1500 μm, preferably between 200 μm and 1000 μm.

The values x10, x50 and x90 are based on the mass of the particles, which means that the particles with a particle size above the x50 value constitute 50 wt. % of the agglomerate, while the remaining 50 wt. % of the agglomerate comprises particles with a particle size smaller than x50. Accordingly, the agglomerate contains 10 wt. %

of particles with a particle size below the x10 value and 10 wt. % above the x90 value.

Preferred agglomerates have a flowability [ffc] of greater than 4, preferably greater than 8 and in particular greater than 10 (free-flowing). Typically, the [ffc] value is less than 30, however higher values are not detrimental. The bulk density of preferred agglomerates is greater than 200 g/L. In particular, the bulk density should be at least in the range of pure ground creatine. Therefore, agglomerates with a bulk density between 250 g/L and 1,000 g/L are particularly preferred. However, higher bulk densities are not detrimental. To facilitate pouring the agglomerates into a beverage, a low angle of repose is advantageous. Preferred agglomerates have an angle of repose of less than 50°, in particular less than 45°. Typically, the angle of repose is greater than 25°, but smaller angles of repose are not detrimental. The pourability of preferred agglomerates is at least satisfactory (i.e. the agglomerate trickles through a vessel outlet having a diameter of 8 mm). Furthermore, preferred agglomerates have a lower dustiness than ground creatine. The dust number is preferably less than 25, in particular between 2 and 20 or between 3 and 15. From these parameters it can be seen that the agglomerates described herein have excellent handling properties. They can be easily poured into beverages and have good wettability in water.

In addition, the agglomerates described herein dissolve quickly and with little residue in an aqueous environment, e.g. in beverages. The dissolution rate approaches or even exceeds that of ground creatine.

The agglomerates according to the invention can be produced, for example, by granulation in mixers with high shear forces, by agglomeration in the fluidized bed or by extrusion. The properties of the agglomerates obtained differ somewhat depending on the manufacturing process.

When granulating in the mixer, a free-flowing agglomerate ([ffc]>10) with acceptable solubility characteristics but with good pourability and good bulk properties is obtained (very good bulk density >400 g/L and good angle of repose <40°. In addition, the granules have a very low dustiness (dust number <12, usually even <10).

When agglomerating in the fluidized bed, easily flowing ([ffc]>4), usually even free-flowing ([ffc]>10) agglomerates with satisfactory pourability are obtained. However, the fluidized bed agglomerates usually exhibit very good solubility characteristics, which is even better than that of ground creatine. In addition, the bulk properties of the fluidized bed agglomerates are good (bulk density >200 g/L, in particular between 250 g/L and 600 g/L, good angle of repose <45°, in particular <40°. The fluidized bed agglomerates are also characterized by low dustiness (dust number <20, in particular <15).

The extrudates are also free-flowing ([ffc]>10) and have good pourability. The bulk properties are very good (bulk density >400 g/L, angle of repose <40°. In addition, the extrudates have low dustiness (dust number <15). The extrudates have very good solubility characteristics, significantly better than that of ground creatine.

The granules have a preferred grain size distribution which is characterized by an x10 value in the range from 10 μm to 800 μm, in particular between 100 μm and 600 μm, an x50 value in the range from 80 μm to 1,200 μm, in particular from 550 μm to 1,000 μm and an x90 value in the range from 200 μm to 1,800 μm, in particular from 800 μm to 1,500 μm.

The fluidized bed agglomerates have a preferred grain size distribution which is characterized by an x10 value in the range from 1 μm to 300 μm, in particular between 5 μm and 100 μm or even between 5 μm and 50 μm, an x50 value in the range from 30 μm to 500 μm, in particular from 50 μm to 300 μm and an x90 value in the range from 100 μm to 1,500 μm, in particular from 200 μm to 800 μm.

The extrudates have a preferred grain size distribution characterized by an x10 value in the range from 100 μm to 800 μm, in particular between 300 μm, preferably 400 μm and 750 μm, an x50 value in the range from 200 μm to 1,200 μm, in particular from 500 μm to 1,000 μm and an x90 value in the range from 300 μm to 1,800 μm, in particular from 800 μm to 1,500 μm.

The agglomerates described herein are particularly suitable as a bulk powder for stirring the creatine they contain into beverages such as mineral water, fruit juices or sweet beverages. However, the agglomerates can also be used as direct agglomerates. In this case, the agglomerate is poured directly onto the tongue and washed down with a beverage if necessary.

### EMBODIMENTS

### I) Test Methods and Properties

### 1. Method for Determining the Dissolution Rate:

In a 250 mL tumbler with an inner diameter of 5.5 cm, 175 ml of water is added at 23° C. and stirred with a glass stirrer at 60 rotations per minute. Then 1.75 g creatine or creatine agglomerate is added and stirred for 10 seconds, then the stirrer is switched off and the suspension is immediately filtered through a ceramic strainer with blue band filter and suction bottle. The moist filter residue is dried. The mass of the filter residue serves as a measure of the dissolution rate; the lower the mass, the better the dissolution rate.

### 2. Determination of Pourability (Drainage Funnel):

The test equipment consists of five test funnels with the same diameter (36 mm inner diameter) and 28 degree tilt, but with different outlet diameters (2.5 mm; 5 mm; 8 mm; 12 mm and 18 mm). A 50 mL sample of creatine or the creatine formulation is filled into the test funnel, whereby the outlet is closed from below so that no material can drain during filling. In the next step, the outlet is opened completely—without shaking the test funnel—so that the entire outlet cross-section is released. The evaluation parameter is the diameter at which the solid trickles through independently and without external influence. The following applies:
Solid trickles through the 2.5 mm outlet: Grade 1
Solid trickles through the 5 mm outlet: Grade 2
Solid trickles through the 8 mm outlet: Grade 3
Solid trickles through the 12 mm outlet: Grade 4
Solid trickles through the 18 mm outlet: Grade 5
Solid does not trickle through the 18 mm outlet: Grade 6

The lower the grade, the better the pourability.

### 3. Determination of the Angle of Repose

The angle of repose was determined according to the method DIN ISO 4324 (1983-12) Surfactants; powders and granules; determination of the angle of repose. The smaller the angle of repose, the better the flow properties.

### 4. Determination of Flowability

Flowability of solids (powders and agglomerates) is determined using the “Evolution Powder Tester” measuring device from PS Prozesstechnik GmbH, Basel, Switzerland. Flowability is shown in the dimensionless number [ff_{c}]. The method used is determination by compression on the Evolution Powder Tester; no time consolidation is carried out. For this purpose, 25 mL of the solid is weighed into the measuring cell and placed in the measuring device. After starting the measurement, the solid is compressed in the measuring cell at a stamping speed of 15 mm per minute and a force (F₁) of 10000 kPa for 30 seconds. The resulting solid compact in the measuring cell is then loaded again with a stamping speed of 10 mm per minute and slowly increasing force (F₂) until the solid compact breaks. The ratio of the force (F₁) for compression and the force (F₂) for breaking the compact corresponds to the flowability [ff_{c}] and is calculated using the formula:

$$Flowability [ {ff}_{c} ] = \frac{{F}_{1}}{{F}_{2}}$$

The following assessment applies to the classification of flowability [ff_{c}]:

### 5. Determination of Dustiness

The dustiness of solids (powders and agglomerates) is represented in the dimensionless dust number. The dust number is determined using the DustView II dust measuring device from Palas GmbH, Karlsruhe.

Thereby, 30.0 g of a sample is weighed and placed in the funnel on the flap. The measurement is then started by pressing a button on the control panel. The flap opens and the solid falls freely into the dust box. The impact of the solid material whirls up the dust particles. As a result, the light beam emitted by the laser is attenuated by the swirling dust and the attenuated light beam is detected at the receiver. The degree of attenuation (transmission signal) compared to the light beam emitted by the light source is a measure of the dustiness of the solid. A value of 100 means the maximum possible attenuation of the light beam and a value of 0 means no attenuation of the light beam. To determine the dust number, the value of the maximum attenuation of the light beam is added to the value at 30 seconds after the start of the measurement and output as the dust number.

The lower the dust number, the less dusty the solid is.

### 6. Determination of the Bulk Density

The bulk density was determined according to the method DIN ISO 697 (1984-01) surfactants; detergents; determination of bulk density; method by measuring the mass of a given volume.

The higher the bulk density, the more advantageous the handling.

### 7. Determination of the Grain Size Distribution

The grain size distribution was determined using a laser diffraction method on a “HELOS/KR” grain size measuring device from Sympatec GmbH. The R6 measuring aperture was used, which covers a measuring range of 0.5 to 1750 μm. The sample was fed via a vibrating chute with 60% power and a dispersion pressure of 2.5 bar. The software version “WINDOX 5.1.2.0, LD” was used for evaluation. Setting the trigger conditions: Time base 100.00 ms, start at c.opt>=1.0%, validity c.opt from 1.0% to 14.0%, stop at 5.000s c.opt<=0.9% or 10.000s real time. The x10, x50 and x90 values were used to assess the agglomerate quality, represented as the distribution sum Q3 in a histogram of the grain size distribution.

### II) EXAMPLES

Where % indications are given in the examples, these are weight % indications unless explicitly stated otherwise.

### Example 1 (Comparison): Agglomeration in the Eirich Mixer—Use of Modified Starch as a Binder

1500 g of fine creatine monohydrate and 45 g of modified starch (product name: Spezialstärke 6023 FF from Südstärke GmbH) were added to a 10 liter intensive mixer (Eirich). The content of the mixer was then stirred countercurrently at 1500 rpm and 335 g of water was added continuously to the content of the mixer for 3 min. After the water addition was completed, the mixer content was further stirred countercurrently at 1500 rpm and granulation began. After 9 min granulation time, granules in the desired grain size range were obtained. The resulting moist creatine monohydrate granules were dried in a fluidized bed dryer.

### Example 2: Agglomeration in the Eirich Mixer—Use of Maltodextrin 6 as a Binder

1500 g of fine creatine monohydrate was added to a 10 liter intensive mixer (Eirich). Then 42 g of maltodextrin (Glucidex IT 6 from Roquette) dissolved in 450 g of water was added to the creatine monohydrate in the mixer while stirring slowly. The content of the mixer was then stirred countercurrently at 1500 rpm and granulation began. After 7 min granulation time, a granulate in the desired grain size range was obtained. The resulting moist creatine monohydrate granules were dried in a fluidized bed dryer.

### Example 3: Agglomeration in the Eirich Mixer—Use of Maltodextrin 6 as a Binder

1500 g of fine creatine monohydrate was added to a 10 liter intensive mixer (Eirich). Then 75 g of maltodextrin (Glucidex IT 6 from Roquette) dissolved in 400 g of water was added to the creatine monohydrate in the mixer while stirring slowly. The content of the mixer was then stirred countercurrently at 1500 rpm and granulation began. After 15 min granulation time, a granulate in the desired grain size range was obtained. The resulting moist creatine monohydrate granulate was dried in a fluidized bed dryer.

### Example 4 (Comparison): Agglomeration in the Eirich Mixer—Use of Dextrose as a Binder

1500 g of fine creatine monohydrate was added to a 10 liter intensive mixer (Eirich). Then 135 g of dextrose (product name: “Zec+ Dextrose” from Zec+ Nutrition) dissolved in 350 g of water was added to the creatine monohydrate in the mixer while stirring slowly. The content of the mixer was then stirred countercurrently at 1500 rpm and granulation began. After 7 min granulation time, a granulate in the desired grain size range was obtained. The resulting moist creatine monohydrate granules were placed in a fluidized bed dryer and dried in the same way as in Examples 1 to 3. The granules disintegrated again into fine particles; the binding effect of dextrose was not sufficient to obtain stable granules.

### Example 5 (Comparison): Agglomeration in the Eirich Mixer—Granulation of the Composition According to an Example from US 2002/0151593 A1.

3000 g of finely ground dextrose (product name: “Zec+ Dextrose” from Zec+ Nutrition) and 750 g of fine creatine monohydrate were added in a 10 liter intensive mixer (Eirich). Then, 375 g of water was added to the solid mixture in the mixer while stirring slowly. The content of the mixer was then stirred countercurrently at 1500 rpm and granulation began. After 5 min granulation time, granules in the desired grain size range were obtained. The resulting moist creatine monohydrate granules were placed in a fluidized bed dryer and dried in the same way as in Examples 1 to 3. The granules disintegrated again into fine particles; the binding effect of dextrose was not sufficient to obtain stable granules.

### Example 6 (Comparison): Ground, Pure Creatine Monohydrate is Used as a Further Comparison

In the following, the products obtained are characterized, as far as possible, using the methods described under I). The results are summarized in Table 1.

TABLE 1
Results of agglomeration by granulation

| | | Example | | | | |
|---|---|---|---|---|---|---|
| | | 6 | 5 | 1 | | |
| | | (comparison) | (comparison) | (comparison) | 2 | 3 |
| Creatine MH [%] | | 100 | 20.2 | 96.7 | 97.2 | 95.0 |
| Maltodextrin 6 [%] | | | | | 2.7 | 4.8 |
| Modified starch | | | | 2.9 | | |
| [%] | | | | | | |
| Dextrose [%] | | | 79.5 | | | |
| Water [%] | | 0.0 | 0.3 | 0.4 | 0.1 | 0.2 |
| Solubility | | 24.7 | 1.0 | 756 | 109 | 209 |
| characteristics | | | | | | |
| residue [mg] | | | | | | |
| Pourability | | 6 | 6 | 2 | 2 | 2 |
| Bulk density [g/L] | | 270 | 443 | 554 | 549 | 623 |
| Angle of repose [°] | | 54 | 48 | 36 | 36 | 35 |
| Dustiness [dust | | 23.2 | 48.5 | 3.45 | 8.21 | 8.06 |
| number] | | | | | | |
| Flowability [ff_{c}] | | 1.9 | 3.4 | 22.3 | 24.4 | 24.5 |
| Grain size | ×10 value | 1.32 | 6.36 | 135 | 408 | 147 |
| distribution | [μm] | | | | | |
| | ×50 value | 13.0 | 24.0 | 518 | 767 | 669 |
| | [μm] | | | | | |
| | ×90 value | 45.8 | 49.5 | 869 | 1216 | 1146 |
| | [μm] | | | | | |

### Example 7: Agglomeration in the Fluidized Bed—Use of Maltodextrin 6 (Glucidex IT 6 from Roquette) as a binder.

In a fluidized bed agglomeration apparatus, 12.0 kg of fine creatine monohydrate was introduced into the process chamber and the apparatus was then tightly sealed for the agglomeration process. A suitable and preheated air volume flow was then set, which enabled the fluidization of the creatine monohydrate particles in the fluidized bed. Thereby the internal temperature of the apparatus was heated. Once the appropriate volume flow was set, 9.2 kg of a 20% aqueous maltodextrin 6 solution was countercurrently sprayed into the fluidized bed over a period of 25 min using a two-substance nozzle, whereby an agglomerate was formed as the spraying time increased. After the end of spraying, the resulting agglomerate was dried further in the fluidized bed until the water not bound to the creatine as monohydrate was removed.

### Example 8: Agglomeration in the fluidized Bed-Use of Maltodextrin 6 (Glucidex IT 6 from Roquette) as a Binder

In a fluidized bed agglomeration apparatus, 12.0 kg of fine creatine monohydrate was introduced into the process chamber and the apparatus was then tightly sealed for the agglomeration process. A suitable and preheated air volume flow was then set, which enabled the fluidization of the creatine monohydrate particles in the fluidized bed. Thereby the internal temperature of the apparatus was heated. Once the appropriate volume flow was set, 5.6 kg of a 20% aqueous maltodextrin 6 solution was countercurrently sprayed into the fluidized bed over a period of 16 min using a two-substance nozzle, whereby an agglomerate was formed as the spraying time increased. After the end of spraying, the resulting agglomerate was dried further in the fluidized bed until the water not bound to the creatine as monohydrate was removed.

### Example 9: Agglomeration in the Fluidized Bed—Use of Maltodextrin 6 (Glucidex IT 6 from Roquette) as a Binder

In a fluidized bed agglomeration apparatus, 12.0 kg of fine creatine monohydrate was introduced into the process chamber and the apparatus was then tightly sealed for the agglomeration process. A suitable and preheated air volume flow was then set, which enabled the fluidization of the creatine monohydrate particles in the fluidized bed. Thereby the internal temperature of the apparatus was heated. Once the appropriate volume flow was set, 4.8 kg of a 20% aqueous maltodextrin 6 solution was countercurrently sprayed into the fluidized bed over a period of 24 min using a two-substance nozzle, whereby an agglomerate was formed as the spraying time increased. After the end of spraying, the resulting agglomerate was dried further in the fluidized bed until the water not bound to the creatine as monohydrate was removed.

In the following, the products obtained are characterized, as far as possible, using the methods described under I). The results are summarized in Table 2.

TABLE 2
Results of agglomeration in the fluidized bed:

| | | Example | | | | |
|---|---|---|---|---|---|---|
| | | 6 | 5 | | | |
| | | (compar- | (compar- | | | |
| | | ison) | ison) | 7 | 8 | 9 |
| Creatine MH [%] | | 100 | 20.2 | 86.6 | 91.4 | 92.5 |
| Maltodextrin 6 [%] | | | | 13.3 | 8.5 | 7.4 |
| Modified starch [%] | | | | | | |
| Dextrose [%] | | | 79.5 | | | |
| Water [%] | | 0.0 | 0.3 | 0.1 | 0.1 | 0.1 |
| Solubility | | 24.7 | 1.0 | 38.0 | 6.5 | 21.2 |
| characteristics | | | | | | |
| Residue [mg] | | | | | | |
| Pourability | | 6 | 6 | 3 | 3 | 3 |
| Bulk density [g/L] | | 270 | 443 | 280 | 303 | 355 |
| Angle of repose [°] | | 54 | 48 | 38 | 39 | 36 |
| Dustiness [dust | | 23.2 | 48.5 | 10.4 | 11.6 | 11.8 |
| number] | | | | | | |
| Flowability [ff_{c}] | | 1.9 | 3.4 | 10.8 | 10.6 | 10.6 |
| Grain size | ×10 value | 1.32 | 6.36 | 17.1 | 16.3 | 11.9 |
| distribution | [μm] | | | | | |
| | ×50 value | 13.0 | 24.0 | 110 | 89.3 | 141 |
| | [μm] | | | | | |
| | ×90 value | 45.8 | 49.5 | 251 | 245 | 632 |
| | [μm] | | | | | |

### Example 10: Agglomeration by Moisture Extrusion—Use of Maltodextrin 6 (Glucidex IT 6 from Roquette) as a Binder

In an intensive mixer, 16.0 kg of fine creatine monohydrate and 4.7 kg of a 20% maltodextrin 6 solution were homogeneously mixed. The moist powder was then fed into a low-pressure extruder and extruded through a 0.7 mm die, initially resulting in rod-shaped extrudates. The rods were rounded into pellets in a spheronizer and then dried in the fluidized bed.

### Example 11: Agglomeration by Moisture Extrusion—Use of Maltodextrin 6 (Glucidex IT 6 from Roquette) as a Binder

In an intensive mixer, 16.0 kg of fine creatine monohydrate and 4.0 kg of a 7% maltodextrin 6 solution were homogeneously mixed. The moist powder was then fed into a low-pressure extruder and extruded through a 0.7 mm die, initially resulting in rod-shaped extrudates. The rods were rounded into pellets in a spheronizer and then dried in the fluidized bed.

In the following, the products obtained are characterized, as far as possible, using the methods described under I). The results are summarized in Table 3.

TABLE 3
Results of agglomeration by moisture extrusion:

| | | 6 | 5 | | |
|---|---|---|---|---|---|
| Example | | (comparison) | (comparison) | 10 | 11 |
| Creatine MH [%] | | 100 | 20.2 | 94.5 | 98.3 |
| Maltodextrin 6 [%] | | | | 5.5 | 1.7 |
| Modified starch [%] | | | | | |
| Dextrose [%] | | | 79.5 | | |
| Water [%] | | 0.0 | 0.3 | 0.0 | 0.0 |
| Solubility characteristics | | 24.7 | 1.0 | 8.8 | 5.9 |
| Residue [mg] | | | | | |
| Pourability | | 6 | 6 | 2 | 2 |
| Bulk density [g/L] | | 270 | 443 | 595 | 565 |
| Angle of repose [°] | | 54 | 48 | 31 | 35 |
| Dustiness [dust number] | | 23.2 | 48.5 | 7.25 | 10.4 |
| Flowability [ff_{c}] | | 1.9 | 3.4 | 20.3 | 26.1 |
| Grain | ×10 value | 1.32 | 6.36 | 547 | 526 |
| size | [μm] | | | | |
| distribution | ×50 value | 13.0 | 24.0 | 818 | 770 |
| | [μm] | | | | |
| | ×90 value | 45.8 | 49.5 | 1244 | 1148 |
| | [μm] | | | | |

### Example 12: Comparison of the Dissolution Rate or Solubility Characteristics of Carious Products Containing Creatine Monohydrate

The solubility characteristics of 1.75 g creatine or 1.75 g creatine agglomerate are determined according to the test method described in section I) 1. The results are shown in Table 4.

TABLE 4

| | Undissolved | |
|---|---|---|
| | portion | |
| Designation | to 1.75 g | |
| Creatine monohydrate, not ground, ×50 | 632.9 | mg |
| = 171 μm (comparison) | | |
| Creatine monohydrate, ground, | 24.7 | mg |
| Starting material for Examples 1 to 11 (comparison) | | |
| Creatine monohydrate, granulated, | 109 | mg |
| according to Example 2 | | |
| Creatine monohydrate, agglomerated; | 6.5 | mg |
| according to Example 8 | | |
| Creatine monohydrate, extruded, | 8.8 | mg |
| according to Example 10 | | |

The finely ground creatine monohydrate has a high dissolution rate, whereas coarsely crystalline creatine monohydrate has a slow dissolution rate. Although the creatine monohydrates according to the invention (Examples 2, 8, 10) are large particles, they exhibit a high dissolution rate combined with low dustiness and good bulk behavior.

### Example 13: Microscopic Tests

The exemplary microscopic images shown in FIG. 1 show that the shape of the creatine particles according to the invention is clearly different from the crystalline form of pure, unground creatine monohydrate.

FIG. 1 shows microscope images of the non-ground creatine monohydrate with a x50 value of 171 μm (FIG. 1a), which is present in large, symmetrical, angular, elongated, almost colorless crystals; of the creatine monohydrate agglomerated according to Example 8, which is present in large, loose, irregular, shapeless arranged, angular, almost colorless particles (FIG. 1b); and the creatine monohydrate agglomerated according to Example 10 (FIG. 1c), which is present in large, compact, rounded, white, shiny particles.

In sum, the examples show that ground creatine monohydrate dissolves more quickly in water than coarsely crystalline creatine monohydrate. The finer the degree of grinding, the better the dissolution rate. At the same time, however, the wetting properties of the ground creatine powder by water and the handling of the powder (dustiness) deteriorate.

By agglomerating the ground creatine in the presence of a suitable binder, such as maltodextrin, the disadvantageous handling properties (e.g. bulk properties, dustiness) can be significantly improved. As can also be seen from the examples, the presence of maltodextrin during agglomeration also significantly improves the mechanical stability of the agglomerates (low dustiness). In particular, the addition of maltodextrin significantly improves the solubility characteristics compared to the non-ground, crystalline creatine. The addition of maltodextrin as a binder therefore improves the quality of the granules and at the same time ensures a high dissolution rate of the creatine monohydrate, which was not to be expected.

1. Water-soluble agglomerate comprising:
a) 45 to 99.9 wt. % of ground creatine and/or ground creatine derivatives and/or ground creatine salts, based on the total weight of the agglomerate, and
b) 0.1 to 30 wt. % of a binder containing at least one oligosaccharide, based on the total weight of the agglomerate.

2. Water-soluble agglomerate according to claim 1, wherein the ground creatine derivative is a creatine hydrate.

3. Water-soluble agglomerate according to claim 1, wherein the ground creatine, creatine derivative or creatine salt has a grain size distribution with a x50 value in the range from 2 μm to 150 μm, a x10 value in the range from 0.01 μm to 20 μm and a x90 value in the range from 15 μm to 250 μm, each based on the mass fraction.

4. Water-soluble agglomerate according to claim 1, wherein binder b) comprises at least 90 wt. % of a carbohydrate mixture, based on the total weight of binder b), the carbohydrate mixture consisting of carbohydrates from the group of monosaccharides, oligosccharides and polysaccharides, and the carbohydrate mixture having an average molecular weight Mₙ in the range from 500 to 10,000 g/mol.

5. Water-soluble agglomerate according to claim 1, wherein the agglomerate comprises binder b) containing 0.5 wt. % to 20 wt. % of maltodextrin, based on the total weight of the agglomerate.

6. Water-soluble agglomerate according to claim 5, wherein the maltodextrin has a dextrose equivalent of 3 to 15.

7. Water-soluble agglomerate according to claim 1, wherein the agglomerate comprises no or less than 5 wt. % of free water.

8. Water-soluble agglomerate according to claim 1, wherein the agglomerate contains, based on the total weight of the agglomerate, 80 wt. % to 99.5 wt. % creatine, creatine derivatives and/or creatine salts.

9. Water-soluble agglomerate according to claim 1, wherein the angle of repose is less than 45° and/or
the bulk density is greater than 200 g/L and/or
the flowability [ffc] is greater than 4, and/or
the dust number is less than 25.

10. Water-soluble agglomerate according to claim 1, wherein the agglomerate is a fluidized bed agglomerate, a granulate, or extrudate.

11. Water-soluble agglomerate according to claim 1, comprising:
a) 45 to 99.9 wt. % of ground creatine and/or ground creatine derivatives and/or ground creatine salts, based on the total weight of the agglomerate; and
b) 0.1 to 30 wt. % of a binder containing at least one oligosaccharide, based on the total weight of the agglomerate; and
c) 0 to 20 wt. % of other additives; and
d) 0 to less than 5 wt. % of free water.

12. A method of applying a water-soluble agglomerate according to claim 1 as a bulk material for dissolution in beverages or as a direct agglomerate.

13. Process for preparing a water-soluble agglomerate according to claim 1, wherein ground creatine is agglomerated in a mixer, in a fluidized bed, or by extrusion in the presence of 0.1 wt. % to 30 wt. % of a binder comprising at least one oligosaccharide, based on the total weight of the agglomerate.

14. Water-soluble agglomerate according to claim 1, wherein the ground creatine derivative is creatine monohydrate.f

15. Water-soluble agglomerate according to claim 1, wherein the agglomerate comprises no or less than 2 wt. % of free water.

16. Water-soluble agglomerate according to claim 1, wherein the agglomerate contains, based on the total weight of the agglomerate, 80 wt. % to 99.5 wt. % creatine monohydrate.

17. Water-soluble agglomerate according to claim 1, comprising at least 60 wt. % of ground creatine and/or ground creatine derivatives and/or ground creatine salts.

18. Water-soluble agglomerate according to claim 1, comprising 1 to 18 wt. % of a binder containing at least one oligosaccharide based on the total weight of the agglomerate.

19. Water-soluble agglomerate according to claim 1, comprising:
a) at least 75 wt. % of ground creatine and/or ground creatine derivatives and/or ground creatine salts, based on the total weight of the agglomerate; and
b) 5 to 15 wt. % of a binder containing at least one oligosaccharide based on the total weight of the agglomerate; and
c) at most 5 wt. % of other additives; and
d) less than 2 wt. % of free water.

20. Water-soluble agglomerate according to claim 19, wherein the ground creatine and/or ground creatine derivatives and/or ground creatine salts comprises creatine monohydrate, and wherein the binder comprises maltodextrin.
